(19) United States
(12) Patent Application Publication
(10) Pub. No.: US 2015/0366623 A1
(43) Pub. Date: Dec. 24, 2015

(54) MEDICAL INSTRUMENT WITH FLEXIBLE WRIST MECHANISM

(71) Applicant: Intuitive Surgical Operations, Inc., Sunnyvale, CA (US)

(72) Inventor: Stephen J. Blumenkranz, Redwood City, CA (US)

(21) Appl. No.: 14/842,202

(22) Filed: Sep. 1, 2015

Related U.S. Application Data
(63) Continuation of application No. 13/210,196, filed on Aug. 15, 2011, now abandoned.

(51) Int. Cl. A61B 19/00

(52) U.S. Cl. CPC A61B 19/22; A61B 2017/00309

(57) ABSTRACT

A medical instrument includes a unitary wrist structure including a first connector portion having an upper stop surface, a second connector portion having a lower stop surface, and a compact flexure integral therewith. The first and second connector portions are coupled together by the compact flexure. Deflection δ of the compact flexure is characterized by the formula δ=(PL³/3EI)+(PL/kAG), wherein P is a load force applied to the compact flexure, L is a beam length of the compact flexure, E is Young's modulus of the compact flexure, I is a moment of inertia of the compact flexure, k is a shear factor for a cross-section of the compact flexure, A is a cross-section area of the compact flexure, and G is a shear modulus of the compact flexure.

### PRIORITY DATA

This is a continuation of U.S. patent application Ser. No. 13/210,196, filed on Aug. 15, 2011, entitled “Medical Instrument With Flexible Wrist Mechanism,” the disclosure of which is incorporated hereby in its entirety.

### TECHNICAL FIELD

The present invention relates generally to a medical instrument, and more particularly to a medical instrument component for holding a mechanism attached therein in different positions and orientations.

### BACKGROUND ART

Modern tools and manipulating instruments, especially instruments with jaws for performing surgical operations, such as cutting, grasping and holding, are providing increasing levels of functionality and strength to support modern needs including applications in minimally invasive surgery. It is desirable to further reduce the diameter of these instruments to reduce incision size and post-operative pain and scarring and to address smaller anatomy in pediatric, vascular and nerve surgery, and in micro-surgery such as ophthalmic surgery. However, the mechanisms available for positioning and orienting the jaws of smaller manipulating instruments are not efficient and often lack precision.

In cable actuated hinge mechanisms using pins or shafts on which portions of the hinge pivot, the cable force increases the pivot pin friction resisting hinge rotation. The pivot pin friction will resist movement until sufficient actuating force is applied to overcome the friction. This means greater actuating force than desired must be applied in order to initiate hinge rotation. This undesirable situation can cause excessive motion as the friction force reduces dramatically once motion is initiated.

Since the hysteresis, usefully thought of as lost motion or wasted energy, of any mechanism varies with the product of the mechanism friction multiplied by its drive train compliance, the combined effect of these friction and compliance increases is a large increase in wrist motion hysteresis as the cross-sectional diameter of an instrument, such as a gripper, decreases for a given type of hinge mechanism. This is particularly detrimental when there is rubbing friction with higher starting friction that results in uneven or unpredictable motion effects sometimes called stiction. Therefore, in order to enable smaller smoothly functioning surgical instrument wrists, it is also desirable to have a new wrist mechanism with lower friction.

The need to reduce costs, to improve efficiencies and performance, and to meet competitive pressures adds an even greater urgency to the critical necessity for finding answers to these problems. Solutions to these problems have been long sought but prior developments have not taught or suggested any solutions and, thus, solutions to these problems have long eluded those skilled in the art.

### DISCLOSURE OF THE INVENTION

The present invention provides a medical instrument that includes: a unitary wrist structure having a first connector portion having a lower stop surface, a compact flexure integral with the first connector portion, and a second connector portion integral with the compact flexure with the second connector portion having an upper stop surface integral with the compact flexure below the lower stop surface and forming an angle with the lower stop surface.

Certain embodiments of the invention have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS

FIG. 1 is a medical instrument with a unitary wrist structure in a first embodiment of the present invention.

FIG. 2 is an enlarged detailed isometric view of the unitary wrist structure in the first embodiment.

FIG. 3 is an enlarged front view of the unitary wrist structure in the first embodiment.

FIG. 4 is an enlarged side view of the unitary wrist structure in the first embodiment.

FIG. 5 is an enlarged side view of the unitary wrist structure in the first embodiment partially flexed forward.

FIG. 6 is an enlarged side view of the unitary wrist structure in the first embodiment partially flexed forward and partially flexed to the left.

FIG. 7 is an enlarged detailed isometric view of a unitary joint structure in a second embodiment.

FIG. 8 is a front view of the unitary joint structure in the second embodiment.

FIG. 9 is a front view of the unitary joint structure in the second embodiment in a fully flexed position.

FIG. 10 is a side view of the unitary joint structure in the second embodiment.

FIG. 11 is an enlarged detailed isometric view of the unitary wrist structure in a third embodiment.

FIG. 12 is an enlarged front view of the unitary wrist structure in the third embodiment.

FIG. 13 is an enlarged side view of the unitary wrist structure in the third embodiment.

FIG. 14 is an enlarged side view of the unitary wrist structure in the third embodiment partially flexed forward.

FIG. 15 is an enlarged side view of the unitary wrist structure in the third embodiment partially flexed forward and partially flexed to the right.

FIG. 16 is an enlarged isometric view of a compact flexure structure in an exemplary embodiment of the present invention.

### BEST MODE FOR CARRYING OUT THE INVENTION

The following embodiments are described in sufficient detail to enable those skilled in the art to make and use the invention. It is to be understood that other embodiments would be evident based on the present disclosure, and that system, process, or mechanical changes may be made without departing from the scope of the present invention.

In the following description, numerous specific details are given to provide a thorough understanding of the invention. However, it will be apparent that the invention may be practiced without these specific details. In order to avoid obscuring the present invention, some well-known devices, instrument configurations, and process steps are not disclosed in detail.

For expository purposes, the term “horizontal” as used herein is defined as the horizontal direction seen when viewing the drawing as indicated by the figure designation of “FIG.”. The term “vertical” refers to a direction perpendicular to the horizontal as just defined. Terms, such as “above”, “below”, “bottom”, “top”, “side” (as in “sidewall”), “higher”, “lower”, “upper”, “over”, and “under”, are defined with respect to the horizontal, as shown in the figures. The term “on” means there is direct contact between the elements described.

Also, in the following description, connected and coupled are used to describe a relationship between two members. The term “connected” means that the two members are physically and directly joined to each other.

Different members can be connected in variety of ways. For example, different members can be connected by being formed adjacent to each other, such as through molding or carving. Also, for example, different members can be connected by being attached together, such as through adhesives, fasteners, welds, or brazing. The term “wrist” means a structure capable of two degrees of freedom, by being able to bend in more than one direction concurrently. The term “joint” means a structure that is capable one degree of freedom, by being able to bend in two directions that are 180 degrees apart.

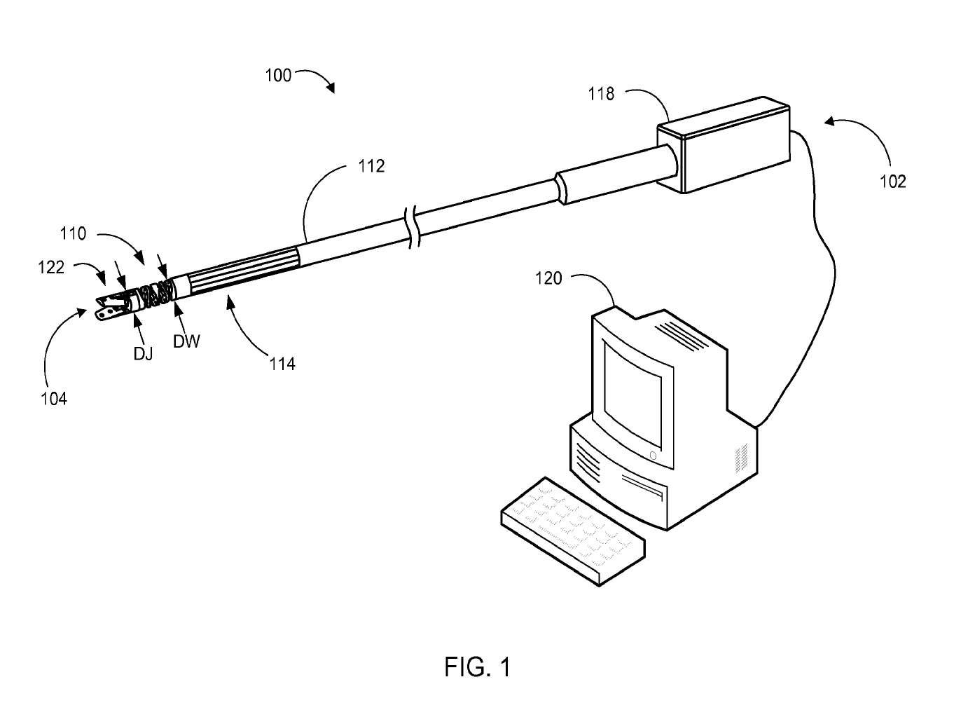

The term “coupled” means that the two members are physically linked through one or more other members. The phrases “reciprocating motion” and “reciprocating movement” are defined to describe a repetitive up-and-down or back-and-forth motion. The phrases “distal” and “proximal” are defined to respectively indicate the directions designated by the related elements in FIG. 1 or along the path of connectivity between the point where the instrument couples to the robot arm (proximal) and the instrument tip that contacts surgical patient tissue (distal).

The drawings showing embodiments of the system are semi-diagrammatic and not to scale and, particularly, some of the dimensions are for the clarity of presentation and are shown exaggerated in the drawing FIGs. Similarly, although the views in the drawings for ease of description generally show similar orientations, this depiction in the FIGs. is arbitrary for the most part. Generally, the invention can be operated in any orientation.

When the instrument diameter is reduced, the diameter of a hinge rotation pulley or hinge lever arm length also decreases and the required cable force increases further. The higher frictional forces and lower mechanical advantage increase cable axial deflection or stretch so that greater movement than desired of the proximal end of the actuating cable is required for a predetermined amount of distal hinge rotation, which means the effective drive train compliance is increased.

Since the hysteresis, usefully thought of as lost motion or wasted energy, of any mechanism varies with the product of the mechanism friction multiplied by its drive train compliance, the combined effect of these friction and compliance increases is a large increase in wrist motion hysteresis as the cross-sectional diameter of an instrument, such as a gripper, decreases for a given type of hinge mechanism. This is particularly detrimental when there is rubbing friction with higher starting friction that results in uneven or unpredictable motion effects sometimes called stiction.

Referring now to FIG. 1, therein is shown a medical instrument 100 with a unitary wrist structure 110 in a first embodiment of the present invention. The unitary wrist structure 110 is a continuous member that bends or provides multi-axis movement to change the relative position and orientation of a member attached thereon. The term “unitary” means a structure made of a single unit of material. The unitary wrist structure 110 is a flexible member that can bend without discrete pivoting pin joints. The unitary wrist structure 110 is also supported by another member attached thereon.

For example, the unitary wrist structure 110 can be connected to a stationary member on one side and a moveable member on the opposite side. The stationary member can hold the unitary wrist structure 110 in position and the unitary wrist structure 110 can be manipulated to position and orient the moveable member.

For a more specific example, the unitary wrist structure 110 can have a mechanical arm attached to one side and a camera, a gripper, or other end effectors on the other side. The mechanical arm can position the unitary wrist structure 110 with the end effector in place. The unitary wrist structure 110 can be manipulated to present different angles, orientations, and views for the camera from the given location.

The unitary wrist structure 110 can be carved or shaped out of a single unit of material, such as plastic or metal alloy. For examples, the unitary wrist structure 110 can be formed by cutting and carving polypropylene plastic or metal alloy or can be formed by using wire electrical discharge machining (EDM) process and post treatment to remove surface layer re-melt, such as when used to shape Nitinol alloy.

The unitary wrist structure 110 can also be molded into shape. For example, the unitary wrist structure 110 can be molded plastic or cast or plated metal. The unitary wrist structure 110 also can be formed as a single injection molding of polypropylene or by metal injection molding (MIM) into a die or mold that has a continuous cavity.

The medical instrument 100 has a proximal end 102 and a distal end 104. The medical instrument 100 can include the unitary wrist structure 110 near the distal end 104, a tube 112 with actuating members 114, and an actuator system 118 at the proximal end 102. The medical instrument 100 can include a jaw mechanism 122 at the distal end.

The jaw mechanism 122 can be at the distal end 104 of the medical instrument 100. The unitary wrist structure 110 can be connected to the jaw mechanism 122. The unitary wrist structure 110 can have the tube 112 attached on the other side. The unitary wrist structure 110 can also be coupled to the actuator system 118 through the actuating members 114. The jaw mechanism 122 can be analogous to a human hand, and the unitary wrist structure 110 can be analogous to a human wrist.

The jaw mechanism 122 can be a mechanical assembly, such as a gripper or a cutter, or a flexibly integral structure manufactured from a single material as a single unit. In an alternate embodiment, the jaw mechanism 122 and the unitary wrist structure 110 can together be continuous and integral. The jaw mechanism 122 and the unitary wrist structure 110 can both be manufactured from a single material as a single unit.

The unitary wrist structure 110 is shown having a cylindrical configuration. The cylindrical configuration eliminates sharp edges and allows the unitary wrist structure 110 and the jaw mechanism 122 to project into tight spaces and move between obstacles such as organs or blood vessels.

It has been discovered that the unitary wrist structure 110 provides for improved manufacturing operations with the advantage of eliminating assembly operations because of its one piece structure. Because of reduced cost, it is feasible to make the unitary wrist structure 110 for single use applications. A single use tool avoids the need for the handling and processing associated with cleaning and resterilization after use, as well as the need for certain instrument design requirements for resterilization, such as the ability to withstand autoclaving.

The tube 112 holds the unitary wrist structure 110 at a location in space. For example, the tube 112 can be a straight tube of a medical instrument.

For illustrative purposes, the tube 112 is shown as a hollow cylindrical member encasing the actuating members 114 within the tube 112. However, it is understood that the tube 112 can be different and have various cross-sectional shapes including interlocking actuating rods, or be solid and have externally the actuating members 114.

The unitary wrist structure 110 is attached at the distal end 104 of the tube 112 and the actuator system 118 at the proximal end 102. Generally, the jaw mechanism 122 is attached at the distal end 104.

In one embodiment, the jaw mechanism 122 has a diameter, depicted as DJ, and the unitary wrist structure 110 has a diameter DW, as depicted in FIG. 1. In one embodiment, DW is equal to DJ so that both portions of the unitary wrist structure 110 can enter easily through the same cannula in an incision during a minimally invasive surgical operation.

The actuator system 118 exerts forces coupled by the actuating members 114 to bend the unitary wrist structure 110 and to actuate the jaw mechanism 122. The actuating members 114, for example, can be a rod or cable or cable and pulley system that is pushed or pulled to bend the unitary wrist structure 110 along the direction of applied force. The actuator system 118 can also be coupled through the actuating members 114 to convey the forces to cause rotating reciprocation motion of the jaw mechanism 122.

The actuator system 118 may include or may be coupled to electrical, hydraulic, or pneumatic power systems to generate the applied forces. A control system 120 can be coupled to the actuator system 118 for controlling the amount of applied forces and motion for the jaw mechanism 122 and the unitary wrist structure 110. The control system 120 is a mechanism that can control the operation of the unitary wrist structure 110. For example, the control system 120 can be a computer and motor assembly or an assembly of handles, gears, and levers.

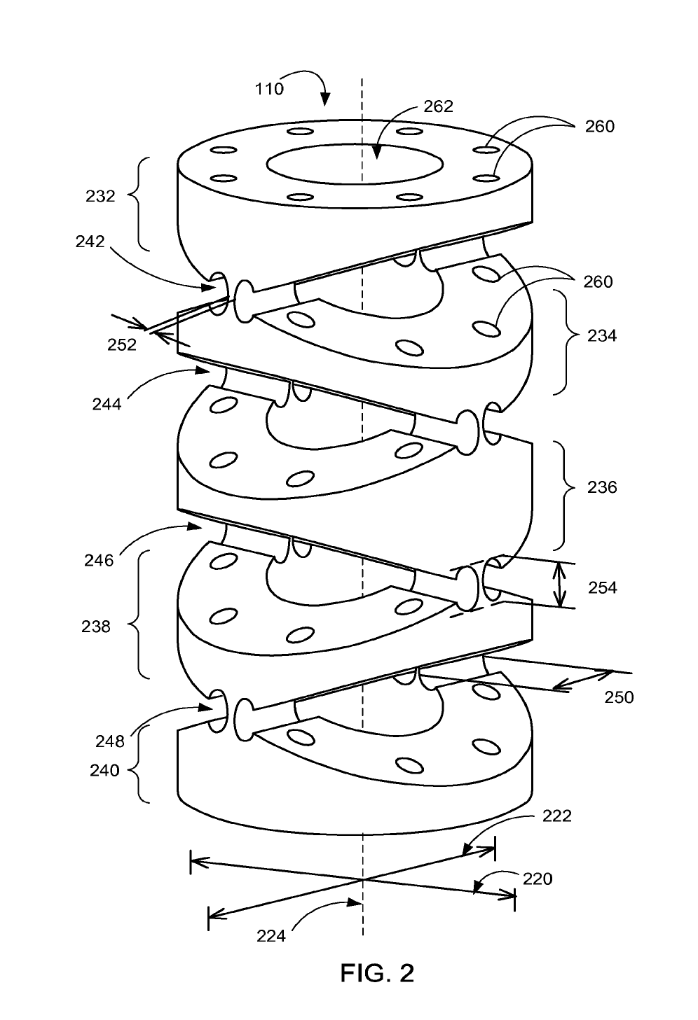

Referring now to FIG. 2, therein is shown an enlarged detailed isometric view of the unitary wrist structure 110 in the first embodiment. The unitary wrist structure 110 has a first transverse dimension 220 and a second transverse dimension 222 along a plane orthogonal to a center line 224.

The first transverse dimension 220 and the second transverse dimension 222 are shown to be the same but do not need to be and may be adjusted based on the geometry of the unitary wrist structure 110. In the case in which they are equal, the unitary wrist structure 110 may be circular in cross section as illustrated in FIG. 2. As an example, the first transverse dimension 220 and the second transverse dimension 222 are shown to be along directions perpendicular to each other but are not necessarily required to be perpendicular.

The unitary wrist structure 110 includes a first connector portion 232, a second connector portion 234, and a third connector portion 236, a fourth connector portion 238, and a fifth connector portion 240. Between the first connector portion 232 and the second connector portion 234 can be a first pair of flexible hinges 242. Between the second connector portion 234 and the third connector portion 236 can be a second pair of flexible hinges 244. Between the third connector portion 236 and the fourth connector portion 238 can be a third pair of flexible hinges 246. Between the fourth connector portion 238 and the fifth connector portion 240 can be a fourth pair of flexible hinges 248. The fifth connector portion 240 can be connectible to the tube 112 of FIG. 1.

Two of the pairs of flexible hinges 242-248 can be placed at right angles to two other of the pairs of flexible hinges 242-248 to allow the first connector portion 232 to be bent in all directions or omni-directionally from the fifth connector portion 240. Further, the pairs of hinges can be arranged so that the bending axes of the outermost hinges, the first pair of flexible hinges 242 and the fourth pair of flexible hinges 248, are parallel to one another. The bending axes of the innermost hinges, the second pair of flexible hinges 244 and the third pair of flexible hinges 246 can be parallel to one another. The unitary wrist structure 110 configured in this way is said to be in an ABBA configuration.

The unitary wrist structure 110 is shown having a Cartesian “yaw-pitch-pitch-yaw” or an ABBA configuration. The configuration is based on the combination of the orientation of the bending axes for the hinges as described above. The terms “yaw” and “pitch” are arbitrary terms, with the “yaw” and “pitch” describing movements in orthogonal directions. The “constant velocity” advantages for the ABBA configuration are described in more detail in U.S. Pat. No. 6,817,974 (filed Jun. 28, 2002), which is incorporated herein by reference.

The pairs of flexible hinges 242-248 can have a flexure width 250 and a flexure thickness 252. The flexure width 250 is a measure of the width of the pairs of flexible hinges 242-248 along the bending axes of each pair of flexible hinges.

For example, as shown in FIG. 2, the bending axes for the first pair of flexible hinges 242 and the fourth pair of flexible hinges 248 are both parallel to the second transverse dimension 222. The flexure width 250 of the first pair of flexible hinges 242 and the fourth pair of flexible hinges 248 can be measured along lines parallel to the second transverse dimension 222. Similarly, the flexure width 250 of the second pair of flexible hinges 244 and the third pair of flexible hinges 246 can be measured along lines parallel to the first transverse dimension 220.

The flexure thickness 252 is a measure of the thickness of the pairs of flexible hinges 242-248. The flexure thickness 252 can be the measure at the thinnest point of the pairs of flexible hinges 242-248. For example, if the pairs of flexible hinges 242-248 have two elliptical concave surfaces opposing each other as shown in FIG. 2, the flexure thickness 252 can be the measure between the vertexes, or the midpoint of each of the concave surfaces.

The flexure thickness 252 can also be measured along a line perpendicular to both the bending axis of each of the pairs of flexible hinges 242-248 and the center line 224. For example, as shown in FIG. 2, the bending axes for the first pair of flexible hinges 242 and the fourth pair of flexible hinges 248 are both parallel to the second transverse dimension 222. Thus, the flexure thickness 252 of the first pair of flexible hinges 242 and the fourth pair of flexible hinges 248 can be measured along lines parallel to the first transverse dimension 220. Similarly, the flexure thickness 252 of the second pair of flexible hinges 244 and the third pair of flexible hinges 246 can be measured along lines parallel to the second transverse dimension 222.

The pairs of flexible hinges 242-248 can also have a flexure length 254. The flexure length 254 is a measure of the length or the height of the pairs of flexible hinges 242-248 along a direction parallel to the center line 224. The flexure length 254 can be measured when the pairs of flexible hinges 242-248 are at a neutral position as shown in FIG. 2. The flexure length 254 can be the distance between the points where the pairs of flexible hinges 242-248 are integral with the adjoining connector portions 232-240.

The pairs of flexible hinges as illustrated in FIG. 2 are typical of flexible hinges and compact flexures. A flexure or flexible hinge is defined as a flexible member that couples two other relatively rigid members. The pairs of flexible hinges 242-248 can be flexible members coupling the connector portions 232-240 that are relatively rigid members.

The pairs of flexible hinges 242-248 can have the flexure thickness 252 that is sufficiently less than the flexure length 254 for providing a bending compliance between the coupled rigid members allowing one rigid member to rotate with respect to the other about an axis along the flexure width 250. The bending axis is located at the midpoint of the flexure length 254 and the flexure thickness 252 when the flexure is straight.

The flexure length 254 and the flexure thickness 252 can have a ratio of length to thickness such that the bending strain and the resulting stress in the flexure are within limits based on its material properties, such as yield strength or fatigue strength, its angular range of motion, and the required flexing motion cycles. The pairs of flexible hinges 242-248 may rigidly transmit forces along at least the flexure width 250 and the flexure length 254 between the two coupled members.

It has been discovered that the pairs of flexible hinges 242-248, when moving, have only low internal hysteresis losses in the material, also called equivalent friction, which are significantly lower than the corresponding actual friction losses in a similarly loaded pin jointed hinge. Hysteresis loss is the loss of motion or energy in mechanisms and structures due to the material properties and mechanical configuration thereof.

The pairs of flexible hinges 242-248 can also be compact flexures. A compact flexure is a flexure made from a plastic material such as injection molded polypropylene or ultra-high molecular weight polyethylene (UHMW-PE) whose material properties permit a short flexure length while at the same time permitting a high number of flexing motion cycles. For example, the flexure length 254 can be less than twice the flexure thickness 252.

The flex life is defined to be the number of times a flexible hinge 242-248 can be reliably flexed or bent without breaking. The flex life of the pairs of compact flexures 242-248 can be enhanced by providing that the melted plastic flows through the hinge from one coupled rigid member toward the other as the mold fills and by flexing the hinge fully in both directions immediately upon removal from the mold. Because of the reduction in the flexure length 254, in relation to the flexure thickness 252, the pairs of compact flexures 242-248 advantageously provide relatively greater stiffness than conventional flexures with respect to forces between the coupled members in the direction of the flexure thickness 252 as may occur when a transverse load along direction of the flexure thickness 252 or a torsional load about the center line 224 is applied between adjacent pairs of coupled members 232-240. Also advantageously, the molded plastic compact flexure may reduce cost by eliminating multiple separate components and associated assembly labor as well as by using a low cost material.

In addition to plastic or metal injection molding or cutting of plastic or metal by machining methods, including metal cutting by wire electrical discharge machining (EDM), the pairs of flexible hinges 242-248 may be configured with sufficient length and uniform thickness to permit exploitation of a planar photo-lithographic plated metal alloy fabrication process by orienting the flexible hinges in the plane of the plated layers to enable unitary structures of 1 mm diameter or smaller. Thus, the invention enables manufacture of functional medical flexible hinge wrist instruments of unprecedented smaller size and further enables use in surgery on correspondingly smaller anatomy such as re-anastomosis of small blood vessels and nerves or manipulation and repair of structures inside the eye.

The flexible hinge portions can be resilient. Therefore, the release of the actuating members 114 of FIG. 1 where there is no pull or push force on the connector portions 232-240 can cause the connector portions 232-240 to move to a position where the pairs of flexible hinges 242-248 are at a defined neutral position. For example, the pairs of flexible hinges 242-248 can be biased to return to generally a straight and extended formation, substantially parallel to one another or to a predetermined bent shape.

The first connector portion 232, the second connector portion 234, the third connector portion 236, the fourth connector portion 238, and the fifth connector portion 240 all have a plurality of actuator holes 260 that are in the periphery of each connector portion and run parallel to the center line 224.

The actuator holes 260 are shown around the periphery of the unitary wrist structure 110. The actuator holes 260 are aligned through the unitary wrist structure 110. In one aspect, the actuator holes 260 go through each connector portion. In another aspect, some of the actuator holes 260 may pass through only some of the connector portions. In one example, some of the actuator holes 260 may pass through only the connector portions 240, 238 and 236 when they will only be used for actuating members to move connector portions 238 and 236 with respect to the fifth connector portion 240.

The actuating members 114 of FIG. 1 that are used to control the unitary wrist structure 110, in the form of control wires or miniature wound or braided cables or ropes, are threaded through the unitary wrist structure 110 through the actuator holes 260 to each of the connector portions. The tension on the actuating members 114 can cause the unitary wrist structure 110 to bend at the pairs of flexible hinges. Depending on the placement of the wrist control wires in the actuator holes 260 and the relative displacement of the actuating members 114, the unitary wrist structure 110 can bend with two to four degrees of freedom although two is a preferred case.

A central channel 262 extends along the center line 224 of the unitary wrist structure 110 for passage of the one of the actuating members 114 for activating the jaw mechanism 122 connected to the unitary wrist structure 110.

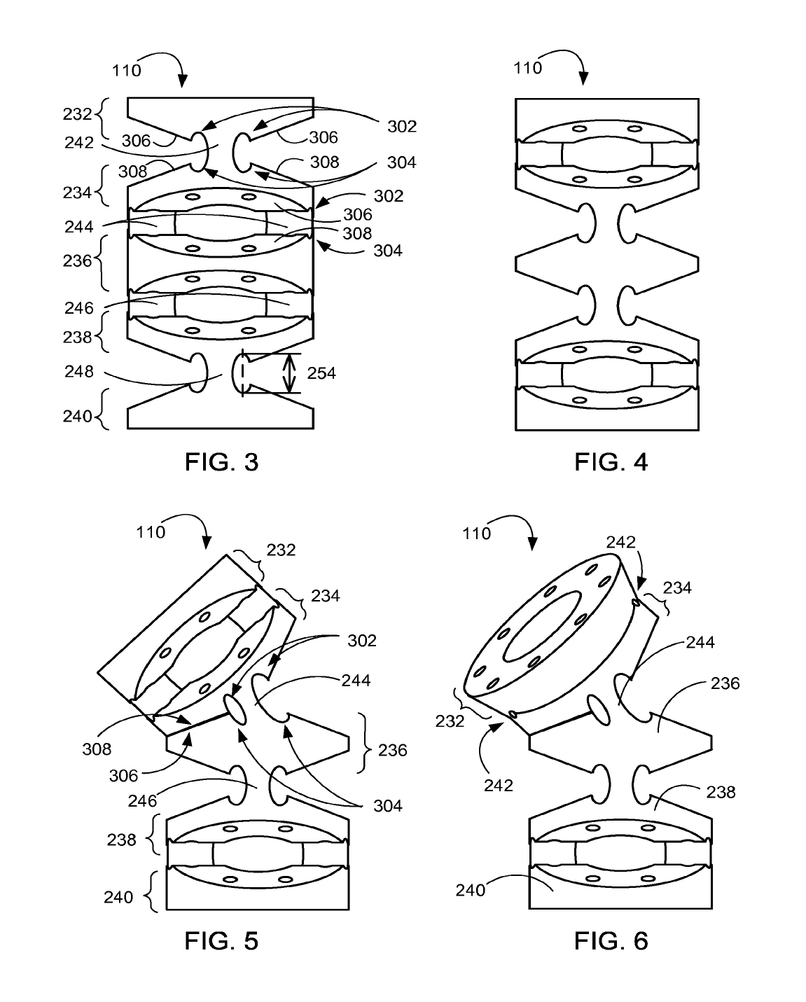

Referring now to FIG. 3, therein is shown an enlarged front view of the unitary wrist structure 110 in the first embodiment. The connector portions 232-238 can have lower relief clearances 302 on the lower portions thereof. The connector portions 234-240 can have upper relief clearances 304 on the upper portions thereof.

The pairs of flexible hinges 242-248 can be between adjacent connector portions. For example, the first pair of flexible hinges 242 can be integral with the lower relief clearances 302 on the lower portion of the first connector portion 232. The first pair of flexible hinges 242 can also be integral with the upper relief clearances 304 on the upper portion of the second connector portion 234. The pairs of flexible hinges 244-248 can similarly provide integral connection between connector portions 234-240.

For illustrative purposes, the pairs of flexible hinges 242-248 are shown connecting to the lower relief clearances 302 at the upper ends of each hinge and the upper relief clearances 304 at the lower ends of each hinge. However, it is understood that the unitary wrist structure 110 can have a different orientation, thereby changing the relative positions of the lower relief clearances 302 and the upper relief clearances 304 and the hinges.

The lower relief clearances 302 and the upper relief clearances 304 are the indentations in connector portions 232-240 at the junction between the each of the connector portions 232-240 and each of the pairs of flexible hinges 242-248. For example, the lower relief clearances 302 can be radii by the first pair of flexible hinges 242 at the junction with the first connector portion 232.

Continuing with the example, the lower relief clearances 302 and the upper relief clearances 304 can be a portion of an ellipse around the vertex point on the major axis of the ellipse while the midpoint of the flexure is at the co-vertex on the minor axis of the ellipse. As a more specific example, the lower relief clearances 302 and the upper relief clearances 304, together with a surface on each of the hinges can form a portion of a cylinder having an elliptical cross-section. The lower relief clearances 302 and the upper relief clearances 304 allow a reduction in the vertical height of the unitary wrist structure 110. The lower relief clearances 302 and the upper relief clearances 304 can be integral with the pairs of flexible hinges 242-248 at the ends of the major axes of the elliptical cross-section. The point for dividing the portions of the hinges and the clearances can be the vertex points of the elliptical cross-section. Further, the flexure length 254 of each hinge can be the length of the major axes of the elliptical cross-section, measured from one vertex to another along a line parallel to the center line 224 of FIG. 2.

The connector portions 232-238 can have lower stop surfaces 306 on the lower portions thereof. The connector portions 234-240 can have upper stop surfaces 308 on the upper portions thereof. The lower relief clearances 302 can be adjacent to the lower stop surfaces 306 and the upper relief clearances 304 can be adjacent to the upper stop surfaces 308. The lower stop surfaces 306 are planar surfaces on the lower portions of the connector portions 232-238 for restricting the movement of the connector portions 232-238 and the bend of the pairs of flexible hinges 242-248. The upper stop surfaces 308 are planar surfaces on the upper portions of the connector portions 232-238 for restricting the movement of the connector portions 234-240 and the bend of the pairs of flexible hinges 242-248.

For illustrative purposes, the lower stop surfaces 306 are shown as being the under surfaces of the connector portions 232-238 and the upper stop surfaces 308 as being on the upper surfaces of the connector portions 234-240. However, it is understood that the unitary wrist structure 110 can be oriented in different positions, thereby changing the relative positions of the lower stop surfaces 306 and the upper stop surfaces 308.

The first connector portion 232 can have a pair of the lower stop surfaces 306 only. The fifth connector portion 240 can have a pair of the upper stop surfaces 308 only. The connector portions 234-238 can have both a pair of the lower stop surfaces 306 and a pair of the upper stop surfaces 308.

For the second connector portion 234, the third connector portion 236, and the fourth connector portion 238, each can have the upper stop surfaces 308 and the lower stop surfaces 306 positioned on top of each other and offset by 90 degrees about the center line 224, or offset by a different angle. For example, the second connector portion 234 is shown having the upper stop surfaces 308 on the left and right side of the second connector portion 234 in the current embodiment. The lower stop surfaces 306 are shown arranged with 90 degree offset and are positioned on the front and the back side.

Continuing with the example, the third connector portion 236 is shown having both the upper stop surfaces 308 and the lower stop surfaces 306 arranged directly on top of each other without angular offset about the center line 224. Both the upper stop surfaces 308 are shown positioned on the front and the back side (not shown) of the unitary wrist structure 110.

The lower stop surfaces 306 of one connector portion can be directly over, above, facing, or minoring the upper stop surfaces 308 of the connector portion below. For example, the lower stop surface 306 of the first connector portion 232 can be directly over and above and mirroring the upper stop surfaces 308 of the second connector portion 234. Also, for example, the second connector portion 234 can have the lower stop surface 306 directly over and mirroring the upper stop surface 308 of the third connector portion 236.

The lower stop surfaces 306 and the upper stop surfaces 308 can be angled away from a horizontal plane for restricting the movement of the connector portions 232-238 and the bend of the pairs of flexible hinges 242-248. The lower stop surfaces 306 can connect to the lower relief clearances 302 of the pairs of flexible hinges 242-248 and extend upwards and away from the pairs of flexible hinges 242-248 at a predetermined angle. The upper stop surfaces 308 can connect to the upper relief clearances 304 of the pairs of flexible hinges 242-248 and extend downwards and away from the pairs of flexible hinges 242-248 at a predetermined angle.

Referring now to FIG. 4, therein is shown an enlarged side view of the unitary wrist structure 110 in the first embodiment. The unitary wrist structure 110 can have identical shapes between front and back and between left and right.

For purposes of discussion, the unitary wrist structure 110 as shown in FIG. 3 will be discussed as facing left in FIGS. 4-6. In other words, FIGS. 4-6 will be discussed as having the right side of the unitary wrist structure 110 in FIG. 3 illustrated. However, it is understood that FIG. 4 can be the left or the right side of the unitary wrist structure 110.

Referring now to FIG. 5, therein is shown an enlarged side view of the unitary wrist structure 110 in the first embodiment partially flexed forward. The unitary wrist structure 110 is shown having the second pair of flexible hinges 244 fully flexed forward.

The first connector portion 232 and the second connector portion 234 are shown leaning forward. The connector portions 236-240 have the same neutral orientation as in FIG. 4.

The unitary wrist structure 110 is shown having the lower stop surface 306 on the front side of the second connector portion 234 abutting the upper stop surface 308 on the front side of the third connector portion 236. The two abutting surfaces can overlap each other and cover the entire surfaces. The two abutting surfaces stop the second pair of flexible hinges 244 from bending forward further. The unitary wrist structure 110 can fully flex forward by similarly bending the third pair of flexible hinges 246 forward in a similar manner. The medical instrument 100 of FIG. 1 can use the actuating members 114 of FIG. 1 to flex the unitary wrist structure 110.

It has been discovered that the slope of the lower stop surfaces 306 and the upper stop surfaces 308 can be used to limit the amount of bend in the unitary wrist structure 110 without further device or limiting mechanism. The slope of the lower stop surfaces 306 and the upper stop surfaces 308 can thusly eliminate external limiters, such as gear mechanism or limitation feature in software, and internal limiters, such as bumps or stoppers, and simplify manufacturing complexity and reduce manufacturing cost.

The lower relief clearance 302 and the upper relief clearance 304 can form a cylinder having an elliptical cross-section as shown on the front side of the second pair of flexible hinges 244. For example, the lower relief clearance 302 and the upper relief clearance 304 can open up or enlarge the arc portion of the lower relief clearances 302 and the upper relief clearance 304 on the backside of the second pair of flexible hinges 244.

The material in the front side of the second pair of flexible hinges 244 compresses when the second pair of flexible hinges 244 bends forward. The material in the backside of the second pair of flexible hinges 244 stretches when the second pair of flexible hinges 244 bends forward.

The slopes of the abutting pair of the upper stop surface 308 and the lower stop surface 306 can limit the bend of the pairs of flexible hinges. For example, the magnitude of the angle away from a horizontal plane for the upper stop surface 308 can be added with that of the lower stop surface 306. The combined angles can be the maximum amount of bend for a given pair of flexible hinges.

Referring now to FIG. 6, therein is shown an enlarged side view of the unitary wrist structure 110 in the first embodiment partially flexed forward and partially flexed to the right. The unitary wrist structure 110 is shown flexed forward as described above. The unitary wrist structure 110 is shown also similarly flexed partially to the right, as viewed from the front in the reference frame of FIG. 3.

The second connector portion 234 has the same position as shown in FIG. 5. The connector portions 236-240 have the same neutral position as shown in FIG. 4. The first connector portion 232 is shown rotated to its left.

The lower stop surface 306 of FIG. 3 on the left side of the first connector portion 232 can abut the upper stop surface 308 of FIG. 3 on the left side of the second connector portion 234. The first pair of flexible hinges 242 can flex and bend to the left, similar to the second pair of flexible hinges 244 as described above.

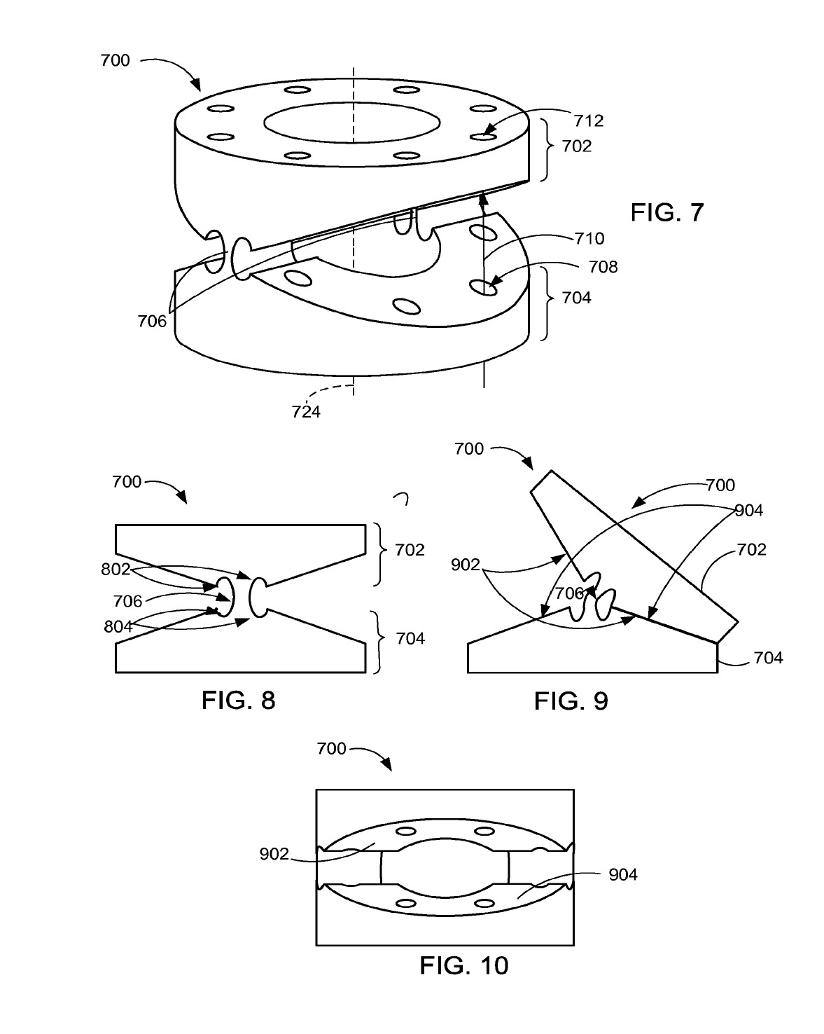

Referring now to FIG. 7, therein is shown an enlarged detailed isometric view of a unitary joint structure 700 in a second embodiment. A first connector portion 702 is similar to the first connector portion 232 of FIG. 2 and a second connector portion 704 is similar to the fifth connector portion 240 of FIG. 2. The first connector portion 702 could be connected to or integral with the jaw mechanism 122 of FIG. 1 and the second connector portion 704 could be connected to the tube 112 of FIG. 1.

Between the first connector portion 702 and the second connector portion 704 are a pair of flexible hinges 706. The pair of flexible hinges 706 is similar to the first pair of flexible hinges 242 of FIG. 2.

The actuating members 114 of FIG. 1 can slide through second actuator holes 708 along a line 710 and can be fastened in first actuator holes 712. By pulling on the actuating members 114 on one side or another of the pair of flexible hinges 706, the unitary joint structure 700 will bend in one direction or another. This principle is also applicable, to a greater extent, to the unitary wrist structure 110 of FIG. 2.

Referring now to FIG. 8, therein is shown a front view of the unitary joint structure 700 in the second embodiment. The pair of flexible hinges 706 connects the first connector portion 702 and the second connector portion 704.

The first connector portion 702 can have lower relief clearances 802 on the lower portion thereof. The second connector portion 704 can have upper relief clearances 804 on the upper portion thereof.

The pair of flexible hinges 706 can be connected to the lower relief clearances 802 at one end and the upper relief clearances 804 at the opposite end. The lower relief clearances 802 and the upper relief clearances 804 can be similar to the lower relief clearances 302 and the upper relief clearances 304 of FIG. 3. The lower relief clearances 802 and the upper relief clearances 804 allow a reduction in the vertical height of the unitary joint structure 700 by permitting the combined connector portions and flexible hinge to fit in a smaller overall height.

Referring now to FIG. 9, therein is shown a front view of the unitary joint structure 700 in the second embodiment in a fully flexed position. The first connector portion 702 can have lower stop surfaces 902 on the lower portion thereof and the second connector portion 704 can have upper stop surfaces 904 on the upper portion thereof. In the fully flexed position, the lower stop surfaces 902 of the first connector portion 702 abut the upper stop surfaces 904 of the second connector portion 704.

The lower stop surfaces 902 can be similar to the lower stop surfaces 306 of FIG. 3. The upper stop surfaces 904 can be similar to the upper stop surfaces 308 of FIG. 3. The lower stop surfaces 902 and the upper stop surfaces 904 are angled to limit the amount of bend of the pair of flexible hinges 706 and of the unitary joint structure 700.

It has been discovered that having the stop surfaces assures the pair of flexible hinges 706 stay within design parameters to assure adequate life of the pair of flexible hinges 706. When the stop surfaces are used in the unitary wrist structure 110 of FIG. 1, it also has been found to prevent the actuating members 114 from bending excessively at the mouths of the actuator holes 260 and causing excessive friction and wear.

The pair of flexible hinges 706 can bend in the middle portion or stretch to accommodate the flexed position. For example, the middle portion of the pair of flexible hinges 706 can stretch on one side and compress on the other side. The lower relief clearances 802 of FIG. 8, the upper relief clearances 804 of FIG. 8 and the stretched surface of the pair of flexible hinges 706 can form a cross-section shaped like the numeral ‘3’ or they can form an elliptical cross section. On the opposite side, the lower relief clearances 802, the upper relief clearances 804 and the compressed surface of the pair of flexible hinges 706 can form a triangular or a heart-shaped cross-section. Also, for example, the pair of flexible hinges 706 can stretch or compress evenly and retain the overall oval shape as exemplified in FIGS. 5 and 6.

Referring now to FIG. 10, therein is shown a side view of the unitary joint structure 700 in the second embodiment. The upper stop surface 904 is shown directly under and minoring the lower stop surface 902.

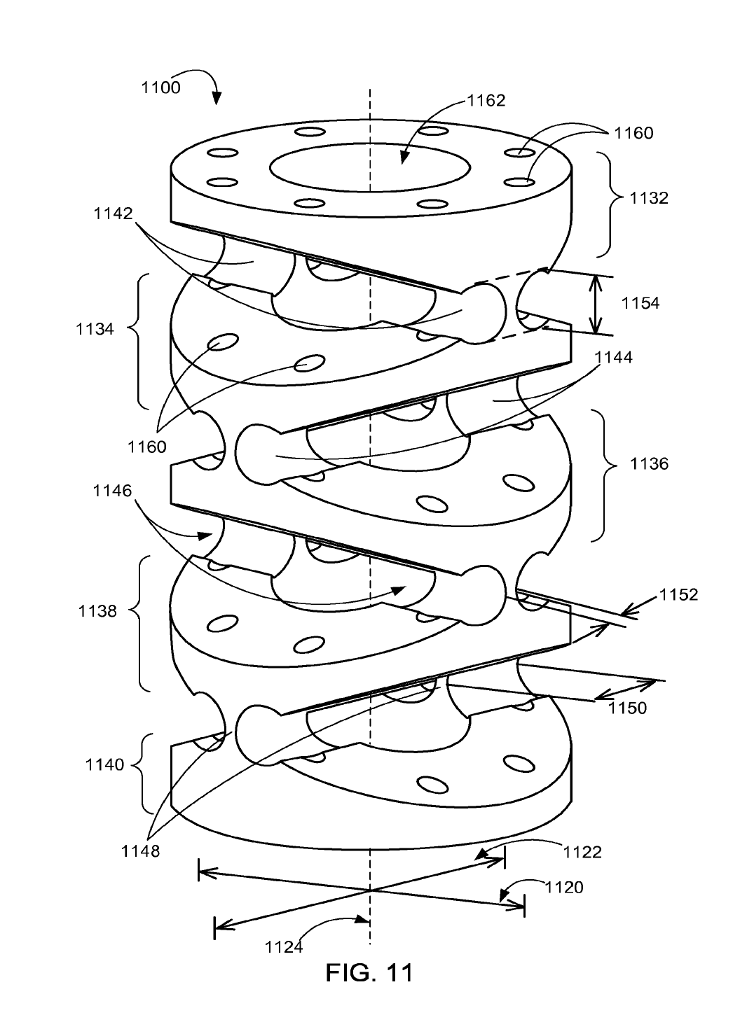

Referring now to FIG. 11, therein is shown an enlarged detailed isometric view of a unitary wrist structure 1100 in a third embodiment. The unitary wrist structure 1100 has a first transverse dimension 1120 and a second transverse dimension 1122 along a plane orthogonal to a center line 1124. The first transverse dimension 1120 and the second transverse dimension 1122 are shown to be the same but do not need to be and may be adjusted based on the geometry of the unitary wrist structure 1100. In the case in which they are equal, the unitary wrist structure 1100 may be circular in cross section as illustrated in FIG. 11.

The unitary wrist structure 1100 includes a first connector portion 1132, a second connector portion 1134, and a third connector portion 1136, a fourth connector portion 1138, and a fifth connector portion 1140. Between the first connector portion 1132 and the second connector portion 1134 is a first pair of flexible hinges 1142. Between the second connector portion 1134 and the third connector portion 1136 is a second pair of flexible hinges 1144. Between the third connector portion 1136 and the fourth connector portion 1138 is a third pair of flexible hinges 1146. Between the fourth connector portion 1138 and the fifth connector portion 1140 is a fourth pair of flexible hinges 1148. The fifth connector portion 1140 is connectible to the tube 112 of FIG. 1.

Two of the pairs of flexible hinges can be placed at right angles to two other of the pairs of flexible hinges to allow the first connector portion 1132 to be bent in all directions or omni-directionally from the fifth connector portion 1140. Further, the pairs of flexible hinges can be arranged alternating 90 degrees so that the bending axes of the first pair of flexible hinges 1142 and the third pair of flexible hinges 1146 are parallel to one another. The bending axes of the second pair of flexible hinges 1144 and the fourth pair of flexible hinges 1148 can be parallel to one another. The unitary wrist structure 1100 configured in this way is said to be in an ABAB configuration.

The unitary wrist structure 1100 is shown having a Cartesian “yaw-pitch-yaw-pitch” or the ABAB configuration. The configuration is based on the combination of the orientation of the bending axes for the hinges as described above. The terms “yaw” and “pitch” are arbitrary terms, with the “yaw” and “pitch” describing movements in orthogonal directions.

By this alternation, further nesting the structural features permits reduction of the length to diameter ratio L/D from L/D=2 in the ABBA case to as little as L/D=1.8 in the ABAB case in one preferred embodiment. In the ABAB embodiment errors in rotational motion of the first connector portion 1132 relative to the fifth connector portion 1140 occur when the fifth connector portion 1140 is rotated about the center line 1124 and the first connector portion 1132 maintains a fixed pointing direction. This is called wrist roll motion. The rotational motion errors of the first connector portion 1132 may be compensated by kinematic computation.

The pairs of flexible hinges 1142-1148 can have a flexure width 1150 and a flexure thickness 1152. The flexure width 1150 is a measure of the width of the pairs of flexible hinges 1142-1148 along the bending axes of each pair of flexible hinges.

For example, as shown in FIG. 11, the bending axes for the first pair of flexible hinges 1142 and the third pair of flexible hinges 1146 are both parallel to the first transverse dimension 1120. The flexure width 1150 of the first pair of flexible hinges 1142 and the third pair of flexible hinges 1146 can be measured along lines parallel to the first transverse dimension 1120. Similarly, the flexure width 1150 of the second pair of flexible hinges 1144 and the fourth pair of flexible hinges 1148 can be measured along lines parallel to the second transverse dimension 1122.

The flexure thickness 1152 is a measure of the thickness of the pairs of flexible hinges 1142-1148. The flexure thickness 1152 can be the measure at the thinnest point of the pairs of flexible hinges 1142-1148. For example, if the pairs of flexible hinges 1142-1148 have two concave surfaces opposing each other as shown in FIG. 11, the flexure thickness 1152 can be the measure between the vertexes of each of the concave surfaces.

The flexure thickness 1152 can also be measured along a line perpendicular to both the bending axis of each of the pairs of flexible hinges 1142-1148 and the center line 1124. For example, as shown in FIG. 11, the bending axes for the first pair of flexible hinges 1142 and the third pair of flexible hinges 1146 are both parallel to the first transverse dimension 1120. Thus, the flexure thickness 1152 of the first pair of flexible hinges 1142 and the third pair of flexible hinges 1146 can be measured along lines parallel to the second transverse dimension 1122. Similarly, the flexure thickness 1152 of the second pair of flexible hinges 1144 and the fourth pair of flexible hinges 1148 can be measured along lines parallel to the first transverse dimension 1120.

The pairs of flexible hinges 1142-1148 can also have a flexure length 1154. The flexure length 1154 is a measure of the length or the height of the pairs of flexible hinges 1142-1148 along a direction parallel to the center line 1124. The flexure length 1154 can be measure when the pairs of flexible hinges 1142-1148 are at a neutral position as shown in FIG. 11. The flexure length 1154 can be the distance between the points where the pairs of flexible hinges 1142-1148 are integral with the adjoining connector portions 1132-1140.

The pairs of flexible hinges as illustrated in FIG. 11 are typical of flexible hinges and compact flexures. A flexure or flexible hinge is defined as a flexible member that couples two other relatively rigid members. The pairs of flexible hinges 1142-1148 can be flexible hinges coupling the connector portions 1132-1140 that are relatively rigid members.

The pairs of flexible hinges 1142-1148 can each have the flexure thickness 1152 that is sufficiently less than the flexure length 1154 for providing a bending compliance between the coupled rigid members allowing one rigid member to rotate with respect to the other about an axis parallel to the flexure width 1150. The bending axis can be located at the midpoint of the flexure thickness 1152 when the flexure is straight.

The flexure length 1154 and the flexure thickness 1152 can have a ratio of length to thickness such that the bending strain and the resulting stress in the flexure are within limits based on its material properties, such as yield strength or fatigue strength limit, angular range of motion, and required flexing motion cycles. The pairs of flexible hinges 1142-1148 may rigidly transmit forces along at least the flexure width 1150 between the two coupled members and preferably along the flexure thickness 1152 and the flexure length 1154.

It has been discovered that the pairs of flexible hinges 1142-1148, when moving, have only low internal hysteresis losses in the material, also called equivalent friction, which are significantly lower than the corresponding actual friction losses in a similarly loaded pin jointed hinge. Hysteresis loss is the loss of motion or energy in mechanisms and structures due to the material properties and mechanical configuration thereof.

The pairs of flexible hinges 1142-1148 can also be compact flexures. A compact flexure is a flexure made from a plastic material such as injection molded polypropylene or ultra-high molecular weight polyethylene (UHMW-PE) whose material properties permit a short flexure length while at the same time permitting a high number of flexing motion cycles. For example, the flexure length 1154 can be less than twice the flexure thickness 1152.

The flex life of the pairs of flexible hinges 1142-1148 can be enhanced by providing that the melted plastic flows through the hinge from one coupled rigid member toward the other as the mold fills and by flexing the hinge fully in both directions immediately upon removal from the mold. Because of its reduced length in relation its thickness, the pairs of flexible hinges 1142-1148 advantageously provide relatively greater stiffness than conventional flexures with respect to forces between the coupled members in the direction of its thickness and with respect to torques about the center line 1124. Also advantageously, the compact flexure may reduce cost by eliminating multiple separate components and associated assembly labor as well as by using a low cost material.

In addition to plastic or metal injection molding or cutting of plastic or metal by machining methods, including metal cutting by wire EDM, the pairs of flexible hinges 1142-1148 may be configured with greater length and uniform thickness to permit exploitation of a planar photo-lithographic plated metal alloy fabrication process by orienting the flexible hinges in the plane of the plated layers to enable unitary structures of 1 mm diameter or smaller. Thus, the invention enables manufacture of functional medical flexible hinge wrist instrument of unprecedented smaller size and further enables use in surgery on correspondingly smaller anatomy such as re-anastomosis of small blood vessels and nerves or manipulation and repair of structures inside the eye.

The first connector portion 1132, the second connector portion 1134, the third connector portion 1136, the fourth connector portion 1138, and the fifth connector portion 1140 all have a plurality of actuator holes 1160 that are in the periphery of each connector portion and run parallel to the center line 1124.

The actuator holes 1160 are shown around the periphery of the unitary wrist structure 1100. The actuator holes 1160 are aligned through the unitary wrist structure 1100. In one aspect, the actuator holes 1160 go through each connector portion. In another aspect, some of the actuator holes 1160 may pass through only some of the connector portions. In one example, some of the actuator holes 1160 may pass through only connector portions 1140, 1138 and 1136 when they will only be used for actuating members to move connector portions 1138 and 1136 with respect to the fifth connector portion 1140.

The actuating members 114 of FIG. 1 that are used to control the unitary wrist structure 1100, in the form of control wires or miniature wound or braided cables or ropes, are threaded through the unitary wrist structure 1100 through the actuator holes 1160 to each of the connector portions. The tension on the actuating members 114 can cause the unitary wrist structure 1100 to bend at the pairs of flexible hinges. Depending on the placement of the wrist control wires in the actuator holes 1160 and the relative displacement of the actuating members 114, the unitary wrist structure 1100 can bend with two to four degrees of freedom although two is a preferred case.

A central channel 1162 extends along the center line 1124 of the unitary wrist structure 1100 for passage of one of the actuating members 114 for activating the jaw mechanism 122 connected to the unitary wrist structure 1100.

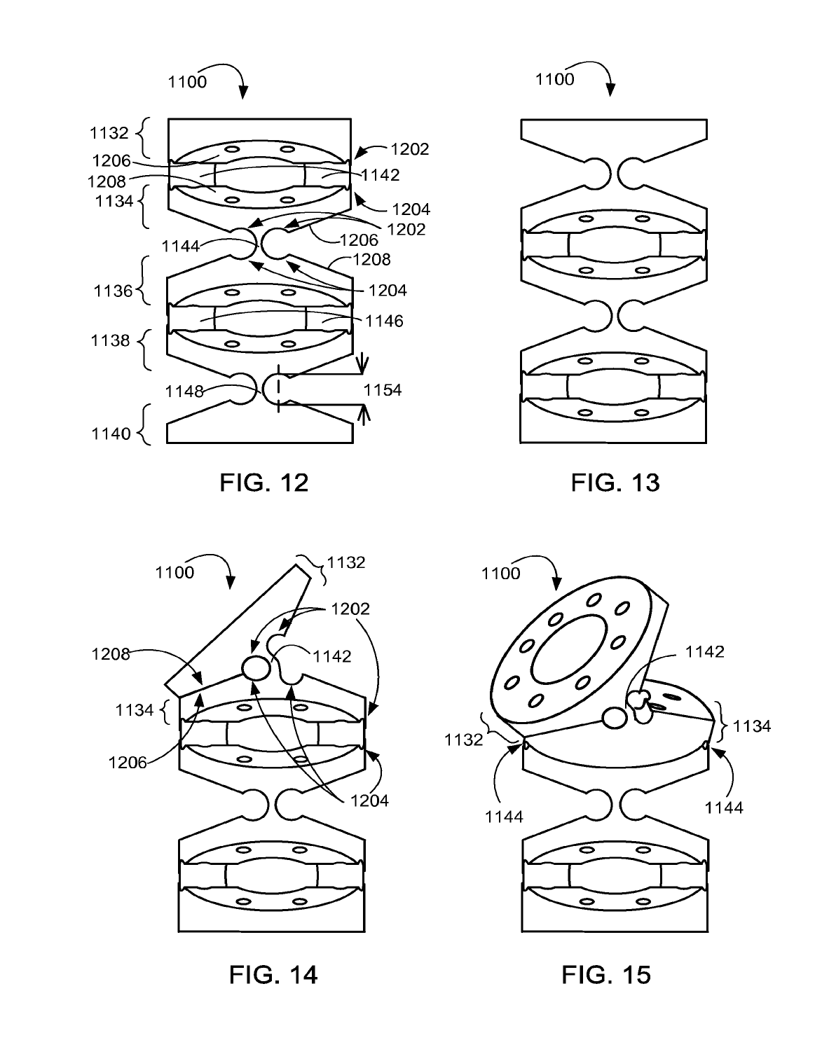

Referring now to FIG. 12, therein is shown an enlarged front view of the unitary wrist structure in the third embodiment. The connector portions 1132-1138 can have lower relief clearances 1202 on the lower portions thereof. The connector portions 1134-1140 can have upper relief clearances 1204 on the upper portions thereof.

The pairs of flexible hinges 1142-1148 can be between adjacent connector portions. For example, the first pair of flexible hinges 1142 can be integral with the lower relief clearances 1202 on the lower portion of the first connector portion 1132. The first pair of flexible hinges 1142 can also be integral with the upper relief clearances 1204 on the upper portions of the second connector portion 1134. The pairs of flexible hinges 1144-1148 can similarly provide integral connection between connector portions 1134-1140.

For illustrative purposes, the pairs of flexible hinges 1142-1128 are shown connecting to the lower relief clearances 1202 at the upper ends of each hinge and the upper relief clearances 1204 at the lower ends of each hinge. However, it is understood that the unitary wrist structure 1100 can have a different orientation, thereby changing the relative positions of the lower relief clearances 1202 and the upper relief clearances 1204 and the hinges. The lower relief clearances 1202 and the upper relief clearances 1204 are the indentations in the connector portions 1132-1140 that are at the junction between the each of the connector portions 1132-1140 and each of the pairs of flexible hinges 1142-1148. For example, the lower relief clearances 1202 can be an arc or a portion of a circular indentation or depression in the first connector portion 1132 at the junction with the first pair of flexible hinges 1142.

Continuing with the example, the lower relief clearances 1202 and the upper relief clearances 1204 can be an arc or a portion of a circular indentation or depression integral with a surface of the opposing pairs of connector portions 1132-1140. The lower relief clearances 1202 and the upper relief clearances 1204, together with a surface of each of the hinges can form a continuous portion of a cylinder having a circular cross-section.

The lower relief clearances 1202 and the upper relief clearances 1204 can be integral with the pairs of flexible hinges 1142-1148 at the ends of the diameter parallel with the center line 1124 of FIG. 11 on the circular cross-section. The point for dividing the portions of the hinges and the clearances can be at the ends of the diameter parallel with the center line on the circular cross-section. Further, the flexure length 1154 of each hinge can be the diameter of the circular cross-section.

The lower relief clearances 1202 and the upper relief clearances 1204 can be connected to lower stop surfaces 1206 and upper stop surfaces 1208. The lower relief clearances 1202 can be connected to the lower stop surfaces 1206 and the upper relief clearances 1204 can be connected to the upper stop surfaces. 1208.

The lower stop surfaces 1206 are planar surfaces on the lower portions of the connector portions 1132-1138 for restricting the movement of the connector portions 1132-1138 and the bend of the pairs of flexible hinges 1142-1148. The upper stop surfaces 1208 are planar surfaces on the upper portions of the connector portions 1134-1140 and minoring the lower stop surfaces 1206 for abutting the lower stop surfaces 1206 to restrict the movement of the connector portions 1132-1138 and the bend of the pairs of flexible hinges 1142-1148.

For illustrative purposes, the lower stop surfaces 1206 are shown as being the lower portion of the connector portions 1132-1138 and the upper stop surfaces 1208 as being the upper portion of the connector portions 1134-1140. However, it is understood that the unitary wrist structure 1100 can be oriented differently, thereby changing the relative positions of the lower stop surfaces 1206 and the upper stop surfaces 1208, the connector portions 1132-1140, and the hinges.

The first connector portion 1132 can have a pair of the lower stop surfaces 1206 only. The fifth connector portion 1140 can have a pair of the upper stop surfaces 1208 only. The connector portions 1134-1138 can have both a pair of the lower stop surfaces 1206 and a pair of the upper stop surfaces 1208.

For the second connector portion 1134, the third connector portion 1136, and the fourth connector portion 1138 each can have the upper stop surfaces 1208 rotationally offset about the center line 1124 from the lower stop surfaces 1206 by 90 degrees or offset by a different angle. For example, the second connector portion 1134 is shown having the upper stop surfaces 1208 on the front and back (not shown) side of the second connector portion 1134 in the current embodiment. The lower stop surfaces 1206 are shown arranged with 90 degree offset and are positioned on the left side and the right side.

The lower stop surfaces 1206 of one connector portion can be directly over, above, facing, or minoring the upper stop surfaces 1208 of the connector portion below. For example, the lower stop surface 1206 of the first connector portion 1132 can be directly over and above and also minor the upper stop surfaces 1208 of the second connector portion 1134. Also, for example, the second connector portion 1134 can have the lower stop surface 1206 directly over and above the upper stop surface 1208 of the third connector portion 1136, with the two sets of surfaces mirroring each other.

The lower stop surfaces 1206 and the upper stop surfaces 1208 can be angled away from a horizontal plane for restricting the movement of the connector portions 1132-1138 and the bend of the pairs of flexible hinges 1142-1148. The lower stop surfaces 1206 can connect to the lower relief clearances 1202 of the pairs of flexible hinges 1142-1148 and extend upwards and away from the pairs of flexible hinges 1142-1148 at a predetermined angle.

Referring now to FIG. 13, therein is shown an enlarged side view of the unitary wrist structure in the third embodiment. The unitary wrist structure 1100 can have identical shapes between front and back and left and right.

For purposes of discussion, the unitary wrist structure 1100 as shown in FIG. 12 will be discussed as facing left in FIGS. 13-15. In other words, FIGS. 13-15 will be discussed as having the right side of the unitary wrist structure 1100 in FIG. 12 illustrated. However, it is understood that FIG. 13 can be the left or the right side of the unitary wrist structure 1100.

Referring now to FIG. 14, therein is shown an enlarged side view of the unitary wrist structure in the third embodiment partially flexed forward. The unitary wrist structure 1100 is shown having the first pair of flexible hinges 1142 fully flexed forward.

The first connector portion 1132 is shown rotated forward. The connector portions 1134-1138 of FIG. 11 have the same neutral orientation as in FIG. 12.

The unitary wrist structure 1100 is shown having the lower stop surface 1206 on the front side of the first connector portion 1132 abutting the upper stop surface 1208 on the front side of the second connector portion 1134. The two abutting surfaces stop the first pair of flexible hinges 1142 from bending forward further. The unitary wrist structure 1100 can fully flex forward by similarly bending the third pair of flexible hinges 1146 of FIG. 12 forward in a similar manner. The medical instrument 100 of FIG. 1 can use the actuating members 114 of FIG. 1 to flex the unitary wrist structure 1100.

It has been discovered that the slope of the lower stop surfaces 1206 and the upper stop surfaces 1208 can be used to limit the amount of bend in the unitary wrist structure 1100 without further device or limiting mechanism. The slope of the lower stop surfaces 1206 and the upper stop surfaces 1208 thusly can eliminate external limiters, such as gear mechanism or limitation feature in software, and internal limiters, such as bumps or stoppers, and simplify manufacturing complexity and reduce manufacturing cost.

The lower relief clearance 1202 and the upper relief clearance 1204 can form a cylinder having a circular cross-section with the front side of the first pair of flexible hinges 1142. The lower relief clearance 1202 and the upper relief clearance 1204 can stretch to form a portion of a cylinder having an elliptical cross-section or a cross-section shaped similar to the numeral ‘3’ on the backside of the first pair of flexible hinges 1142.

The material in the front side of the first pair of flexible hinges 1142 compresses when the first pair of flexible hinges 1142 bends forward. The material in the backside of the first pair of flexible hinges 1142 stretches when the first pair of flexible hinges 1142 bends forward.

The slopes of the abutting pair of the upper stop surface 1208 and the lower stop surface 1206 can limit the bend of the pairs of flexible hinges. For example, the magnitude of the angle away from a horizontal plane for the upper stop surface 1208 can be added with that of the lower stop surface 1206. The combined angle can be the maximum amount of bend for a given pair of flexible hinges.

Referring now to FIG. 15, therein is shown an enlarged side view of the unitary wrist structure in the third embodiment partially flexed forward and partially flexed to the right. The unitary wrist structure 1100 is shown flexed forward as described above. The unitary wrist structure 1100 is shown also similarly flexed partially to the left.

The first connector portion 1132 has the same position relative to the second connector portion 1134 as shown in FIG. 14. The connector portions 1136-1140 of FIG. 11 have the same neutral position as shown in FIG. 12. The first connector portion 1132 and the second connector portion 1134 are shown rotated to the left.

The lower stop surface 1206 of FIG. 12 on the left side of the first connector portion 1132 can abut the upper stop surface 1208 of FIG. 12 on the left side of the second connector portion 1134. The first pair of flexible hinges 1142 can flex and bend to the left, similar to the second pair of flexible hinges 1144 as described above.

The flexible hinges 242-248 of FIG. 2, 706 of FIGS. 7, and 1142-1148 of FIG. 11 largely eliminate the pivot pin friction torques. The combined effect is to greatly reduce hysteresis manifested as lost or unpredictable motion in the unitary wrist structures 110 and 1100 as well as the unitary joint structure 700.

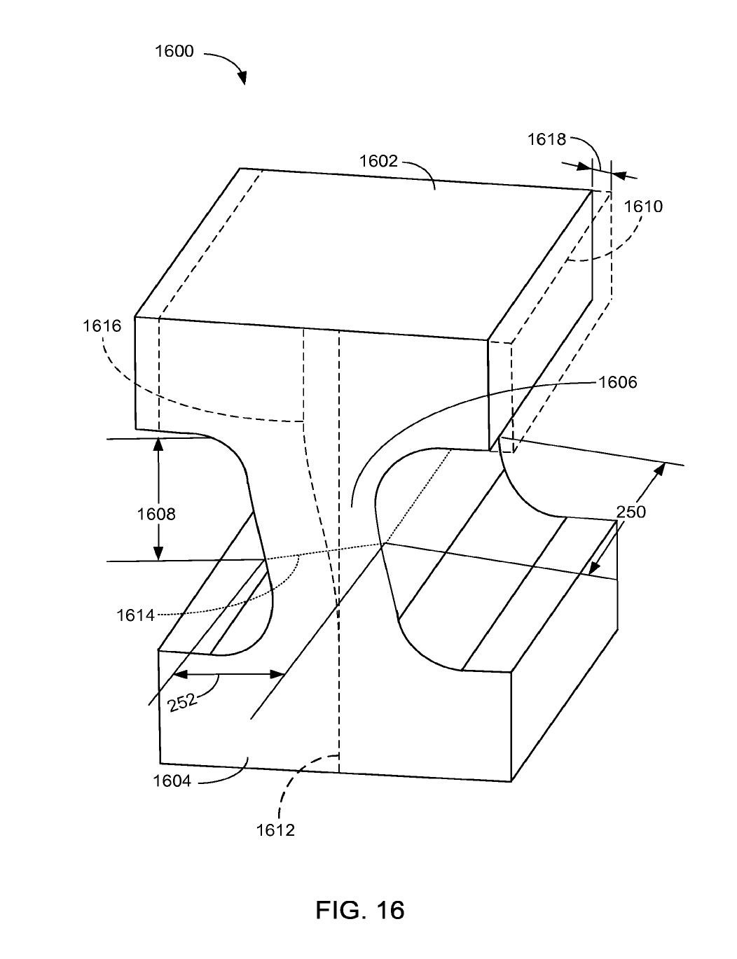

Referring now to FIG. 16, therein is shown an enlarged isometric view of a compact flexure structure 1600 in an exemplary embodiment of the present invention. The enlarged isometric view of the compact flexure structure 1600 depicts a first connection portion 1602 and a second connection portion 1604 having a compact flexure 1606 connected therebetween. A ghost line 1610 indicates the initial location of the first connection portion 1602, which has a resting centerline 1612, prior to the application of a transverse force or a torque, about the axis 224 of FIG. 2, the axis 724 of FIG. 7, or axis 1124 of FIG. 11.

The compact flexure 1606 is a flexible hinge that has been designed to provide substantially equal shear and S-bending displacement or shear dominated displacement when the transverse force or the torque is applied to the application of the compact flexure 1606, such as the unitary wrist structure 110, of FIG. 1. A characteristic of the compact flexure 1606, when subjected to a transverse force or torque, is that the deflection due to an S-bend component 1616 is not more than two times the shear displacement caused by the transverse force or torque.

The compact flexure 1606 is characterized as having a beam length 1608, which represents half of the flexure length 254 of FIG. 2, and having the flexure thickness 252 and the flexure width 250. The measurement of the beam length 1608 from either the first connection portion 1602 or the second connection portion 1604 identifies a mid-plane 1614 of the compact flexure 1606. The mid-plane 1614 represents the position of the inflection point of an S-bend component 1616 of the deformation between the first connection portion 1602 or the second connection portion 1604 when either is moved transversely relative to the other.

The compact flexure 1606 is representative of the design principle of the flexible hinges 242-248 of FIG. 2, 706 of FIGS. 7, and 1142-1148 of FIG. 11 which are defined by opposing concave surfaces rather than flat surfaces. The dimensions of the beam length 1608 and the flexure thickness 252 are designed to provide substantially equal deflection in both shear and S-bending or less deflection in S-bending. The reduced contribution of the S-bending deflection is necessary in order to minimize the torsional and transverse compliance of the unitary wrist structure 110 of FIG. 1 of medical instrument 100 of FIG. 1 while permitting the intended bending of the wrist. A deflection distance is defined to be the sum of the distances that the compact flexure 1606 moves because of shear and S-bending when a transverse force is exerted on the compact flexure 1606, as shown in FIG. 16. The deflection distance may be calculated by the following equation:

$$δ = ( \frac{{PL}^{3}}{3 EI} ) + ( \frac{PL}{kAG} ) \qquad ( 1 )$$

Where δ is half of the deflection 1618, which is the sum of the distance caused by bending and the distance caused by shear. In the bending deflection distance term:

P=load force applied to the member

L=the beam length 1608 as shown in FIG. 16.

E=Young's modulus

I=moment of inertia

In the shear deflection distance term:

P=the load force applied to the member

L=the beam length 1608 as shown in FIG. 16.

k=shear factor=5/6 for rectangular beam cross-section

A=a cross-section area

G=shear modulus

Furthermore,

E=2*G(I+ν) where θ=Poisson ratio

I=b*ĥ3/12 where b is the width 250 and h is the thickness 252 of the beam.

A=b*h

It will be understood by those skilled in the art that by equating the S-bending and shear deflection terms above and eliminating like factors the resulting simplified relation of flexure beam length to thickness is reached

L=h*√(3(1+ν)/5) (2)

It has been discovered that because the length and thickness of compact flexure 1606 are proportioned to have the characteristic that the deflection due to S-bending is substantially equal to or less than the deflection due to shear, caused by a torque applied to the unitary wrist structure 110, of FIG. 2 about axis 224, unitary joint structure 700, of FIG. 7 about axis 724, or unitary wrist structure 1100 about axis 1124 will produce the S-bending that is greatly reduced. This balance between the S-bending displacement and the shear displacement minimizes the excessive rotational compliance and transverse compliance that are present in prior art flexible hinge wrists.

By way of an example, the compact flexure 1606 that is formed of polypropylene which has Poisson ratio ν=0.42 can have equal shear and bending displacement contributions when the flexure beam length 1608 is equal to 0.92 times the flexure thickness 252. It is understood that the beam length 1608 is half of the overall flexure length 254, of FIG. 2. The compact flexure 1606 has the flexure length 254 that is less than twice the flexure thickness 252. The ratio of flexure length to thickness for the compact flexure 1606 with the desired reduced S-bending deflection substantially equivalent to or less than two times the shear deflection may vary somewhat for embodiments with concave flexure 1606 surfaces as in FIGS. 2 thru 15.

Embodiments of the present invention have been found to reduce the number of parts in flexible wrist mechanism to a single integrated part that can be fabricated in a few steps of machining, such as electrical discharge machining or other practical subtractive manufacturing processes.

It has been discovered that the unitary wrist structures 110 and 1100 as well as the unitary joint structure 700 can also be fabricated as a single piece by additive processes such as injection molding from plastic or fiber reinforced plastic composite, metal injection molding followed by sintering or by planar photo-lithographic metal plating thus reducing the part cost and the assembly time. The flexible hinge may be made of an alloy permitting high strains such as a hardened stainless steel, titanium or aluminum alloy or from Nitinol or other more advanced shape memory alloy with even higher permissible strains or from metallic glass materials.

The resulting method, process, apparatus, device, product, and/or system is straightforward, cost-effective, uncomplicated, highly versatile, accurate, sensitive, and effective, and can be implemented by adapting known materials and processes for ready, efficient, and economical manufacturing, application, and utilization.

Another important aspect of the present invention is that it valuably supports and services the historical trend of reducing costs, simplifying systems, and increasing performance.

These and other valuable aspects of the present invention consequently further the state of the technology to at least the next level.

While the invention has been described in conjunction with a specific best mode, it is to be understood that many alternatives, modifications, and variations will be apparent to those skilled in the art in light of the aforegoing description. Accordingly, it is intended to embrace all such alternatives, modifications, and variations that fall within the scope of the included claims. All matters hithertofore set forth herein or shown in the accompanying drawings are to be interpreted in an illustrative and non-limiting sense.

What is claimed is:

1. A medical instrument comprising:
a unitary wrist structure having a plurality of connector portions in a cylindrical configuration, the unitary wrist structure including:
a first connector portion of the plurality of connector portions, the first connector portion having an upper stop surface;
a second connector portion of the plurality of connector portions, the second connector portion having a lower stop surface; and
a compact flexure integral with the first connector portion and the second connector portion and coupling the first connector portion to the second connector portions, wherein a deflection 6 of the compact flexure is characterized by the formula
$$δ = ( \frac{{PL}^{3}}{3 EI} ) + ( \frac{PL}{kAG} )$$
wherein:
P is a load force applied to the compact flexure,
L is a beam length of the compact flexure,
E is Young's modulus of the compact flexure,
I is a moment of inertia of the compact flexure,
k is a shear factor for a cross-section of the compact flexure,
A is a cross-section area of the compact flexure, and
G is a shear modulus of the compact flexure.

2. The medical instrument of claim 1, further comprising:
a first upper relief clearance on a first side of the compact flexure and a second upper relief clearance on a second side of the compact flexure; and
a first lower relief clearance on the first side of the compact flexure and a second lower relief clearance on the second side of the compact flexure.

3. The medical instrument of claim 2, wherein the first upper relief clearance, the first side of the compact flexure, and the first lower relief clearance form a continuous relief surface.

4. The medical instrument of claim 3, wherein the continuous relief surface has an elliptical cross-section having a major axis parallel to the compact flexure.

5. The medical instrument of claim 1, wherein the compact flexure comprises a first side and a second side that are substantially parallel to each other.

6. The medical instrument of claim 5, wherein the compact flexure includes a first region of expanding thickness proximate the first connector portion and a second region of expanding thickness proximate the second connector portion.

7. The medical instrument of claim 5, wherein the compact flexure has a flexure length and a flexure thickness and wherein the flexure length is less than twice the flexure thickness, the flexure thickness being defined between the first side and the second side.

8. The medical instrument of claim 1, the compact flexures is characterized as having an S-bend component of deflection δ that is less than or equal to twice a shear component deflection δ.

9. The medical instrument of claim 1, wherein the first connector portion comprises a coupling surface configured to couple the unitary wrist structure to an end effector or to a mechanical arm.

10. The medical instrument of claim 1, wherein the compact flexure is made from ultra-high molecular weight polyethylene.

11. A medical instrument comprising:
a unitary wrist structure having a plurality of connector portions in a cylindrical configuration, the unitary wrist structure including:
a first connector portion of the plurality of connector portions, the first connector portion having an upper stop surface;
a second connector portion of the plurality of connector portions, the second connector portion having a lower stop surface; and
a compact flexure integral with the first connector portion and the second connector portion and coupling the first connector portion to the second connector portions, wherein the compact flexure is configured such that a deflection δ of the compact flexure is characterized by the formula
$$δ = ( \frac{{PL}^{3}}{3 EI} ) + ( \frac{PL}{kAG} )$$
wherein:
P is a load force applied to the compact flexure,
L is a beam length of the compact flexure,
E is Young's modulus of the compact flexure,
I is a moment of inertia of the compact flexure,
k is a shear factor for a cross-section of the compact flexure,
A is a cross-section area of the compact flexure, and
G is a shear modulus of the compact flexure;
a tube coupled to the unitary wrist structure, wherein the tube includes actuating members extending therethrough, the actuating members also extending through the first connector portion and the second connector portion and being coupled to an actuator system; and
an end effector coupled to the unitary wrist structure and to at least one of the actuating members for actuation by the actuator system.

12. The medical instrument of claim 1, wherein the unitary wrist structure further includes
a first upper relief clearance on a first side of the compact flexure and a second upper relief clearance on a second side of the compact flexure; and
a first lower relief clearance on the first side of the compact flexure and a second lower relief clearance on the second side of the compact flexure, wherein the first upper relief clearance, the first side of the compact flexure, and the first lower relief clearance form a continuous relief surface having an elliptical cross-section.

13. The medical instrument of claim 11, wherein the unitary wrist structure has a first diameter, a distal end of the tube has a second diameter, and a proximal end of the end effector has a third diameter, wherein the second diameter and the third diameter are equal to the first diameter.

14. The medical instrument of claim 13, wherein the first diameter is less than or about 1 mm.

15. The medical instrument of claim 11, wherein the tube comprises a first plurality of actuator holes extending therethrough and the unitary wrist structure comprises a second plurality of actuator holes corresponding to the first plurality of actuator holes extending through the tube.

16. The medical instrument of claim 15, wherein at least one of the plurality of actuating members extends through a first actuator hole of the first plurality of holes and a corresponding actuator hole of the second plurality of actuator holes and is affixed at a distal end of the unitary wrist structure.

17. A unitary wrist structure for coupling an end effector to an elongate hollow cylindrical member, the unitary wrist structure comprising:
a first connector portion of a plurality of connector portions, the first connector portion having an upper stop surface;
a second connector portion of the plurality of connector portions, the second connector portion having a lower stop surface; and
a compact flexure integral with the first connector portion and the second connector portion and coupling the first connector portion to the second connector portions, wherein the compact flexure is coupled to the first connector portion by an upper relief clearance and is coupled to the second connector portion by a lower relief clearance, and wherein the upper relief clearance, a side of the compact flexure, and the lower relief clearance form a continuous relief surface having an elliptical cross-section.

18. The unitary wrist structure of claim 17, wherein the compact flexure is configured so that a deflection δ of the compact flexure is characterized by the formula
$$δ = ( \frac{{PL}^{3}}{3 EI} ) + ( \frac{PL}{kAG} )$$
wherein:
P is a load force applied to the compact flexure,
L is a beam length of the compact flexure,
E is Young's modulus of the compact flexure,
I is a moment of inertia of the compact flexure,
k is a shear factor for a cross-section of the compact flexure,
A is a cross-section area of the compact flexure, and
G is a shear modulus of the compact flexure.

19. The unitary wrist structure of claim 17, wherein the compact flexure has a flexure length and a flexure thickness, wherein the flexure length is less than twice the flexure thickness.

20. The unitary wrist structure of claim 17, wherein the compact flexure is formed by flowing melted plastic through a compact flexure mold and, immediately upon removal from the mold, flexing the compact flexure fully in both directions.
